(19) United States
(12) Patent Application Publication
(10) Pub. No.: US 2010/0004660 A1
(43) Pub. Date: Jan. 7, 2010

(54) BIRTHING AID AND METHOD

(76) Inventors: Daniel D. Powell, Grant, MI (US); Sherry K. Powell, Grant, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/494,766

(22) Filed: Jun. 30, 2009

Related U.S. Application Data
(60) Provisional application No. 61/078,624, filed on Jul. 7, 2008.

(51) Int. Cl. A61B 17/42

(52) U.S. Cl. 606/119; 128/845

(57) ABSTRACT

A birthing aid includes a continuous band of resilient stretchable tubular material, such as surgical tube or an exercise bungee cord. The band includes opposing intermediate sections forming a leg sling, and opposing end sections forming integral loop handles on ends of the leg sling. A soft tubular sheet cover covers the intermediate sections. By this arrangement, the birthing aid is extremely well adapted to comfortably engage and resiliently support a mother's thigh and leg, while also being aesthetically pleasing to the mother while letting the mother focus on child delivery. Further, when properly used, the child delivery is greatly assisted by the birthing aid helping to properly position the mother's pelvic and other bones related to childbirth, and also causing the mother to naturally focus on appropriate muscle action (often called “pulling” versus “pushing”).

This claims benefit under 35 U.S.C. §119(e) of a provisional application Ser. No. 61/078,624, filed Jul. 7, 2008, entitled BIRTHING AID AND METHOD, the entire contents of which are incorporated herein in their entirety.

### BACKGROUND

The present invention relates birthing aids, and methods related to same.

Childbirth is a natural process, but certainly not an easy one. One reason for difficulty is inexperience of the mother in both proper physical position and in proper muscle action. Another reason is that overweight mothers and/or large-framed mothers and/or mothers with inflexible legs and/or mothers with minimal leg strength may have difficulty in holding their legs at an optimal height and angle. In my several years of experience as a nurse in delivery wards, I have not seen or heard of a birthing aid satisfactorily addressing the above issues, in my opinion.

Rhee U.S. Pat. No. 6,668,833 discloses a variety of different birthing aids of interest, including some which use “compliant” materials. However, while the “compliant” materials disclosed in Rhee '833 may flex, it appears that they do not stretch nor “give and take” as the patient naturally adjusts between higher-force and lower-force pulls, nor as the patient adjusts due to fatigue or for comfort. Nor are the illustrated aids in Rhee '833 inviting and “mother-friendly.” Instead, several of the embodiments disclosed in Rhee '833 are relatively complex to place on the mother and/or “awkward” (or uncomfortable) to the mother, such that as a practical matter, many birthing wards will not use them. Still further, some embodiments depend on secure attachment of the Velcro®, which does not always occur. (Notably, where the Velcro® has a high retention force, it is difficult to accurately position and attach it in a comfortable position, and where the Velcro® has a lower retention force, it may unexpectedly release. Further, if used more than once, the attachment strength can be lost by fibers and foreign material entrapped in the Velcro® material.) Some embodiments in Rhee '833 use hard materials, which may or may not fit a mother's hand and which give a hard, cold, unfriendly and uninviting affect. Some embodiments use straps, which are often uncomfortable to the holder's hand when one pulls on them with significant force for a relatively long period of time.

### SUMMARY OF THE PRESENT INVENTION

In one aspect of the present invention, a birthing aid includes a continuous band of resilient stretchable material including opposing intermediate sections forming a leg sling, and opposing end sections forming integral loop handles on ends of the leg sling. A soft sheet cover covers the intermediate section, the band and sheet cover being adapted to engage and support a mother's thigh and leg.

In another aspect of the present invention, a birthing aid includes a band of resilient stretchable material including at least one intermediate section forming a leg sling. End sections form handles on ends of the leg sling. A tubular soft sheet cover covers the at least one intermediate section and is adapted to engage and support a mother's thigh and leg.

In another aspect of the present invention, a birthing aid includes a band of resilient stretchable material including at least one intermediate section forming a leg sling. End sections form handles on ends of the leg sling. A removable soft sheet cover covers the at least one intermediate section and is adapted to engage and support a mother's thigh and leg.

In another aspect of the present invention, a birthing aid includes a continuous band of resilient stretchable material including an intermediate section forming a leg sling. End sections form loops on ends of the leg sling. Soft material is wrapped around the loops to form soft handles providing maximum comfort to a mother in labor that is using the birthing aid.

In another aspect of the present invention, a method of practicing childbirth comprising steps of providing a birthing aid having a resilient stretchable material with an intermediate section forming a leg sling and end sections forming handles; placing the birthing aid under a birthing mother's thigh; and pulling on the end sections in a manner causing the resilient stretchable material to stretch and provide assistance force to hold up the birthing mother's thigh and leg.

These and other aspects, objects, and features of the present invention will be understood and appreciated by those skilled in the art upon studying the following specification, claims, and appended drawings.

### BRIEF DESCRIPTION OF DRAWINGS

FIG. 1 is a perspective view of a birthing mother using a pair of the present birthing aids.

FIG. 2 is a plan view of the birthing aid shown in FIG. 1.

FIGS. 3-4 are cross sections taken along lines III-III and IV-IV in FIG. 2.

FIGS. 5-7 are perspective views of modified birthing aid devices, where material is used to retain two handles of a given device together such as apertured strap and buttons (FIG. 5), strap and buckle (FIG. 6), and strap and hook-and loop patches (FIG. 7).

### DETAILED DESCRIPTION OF PREFERRED EMBODIMENTS

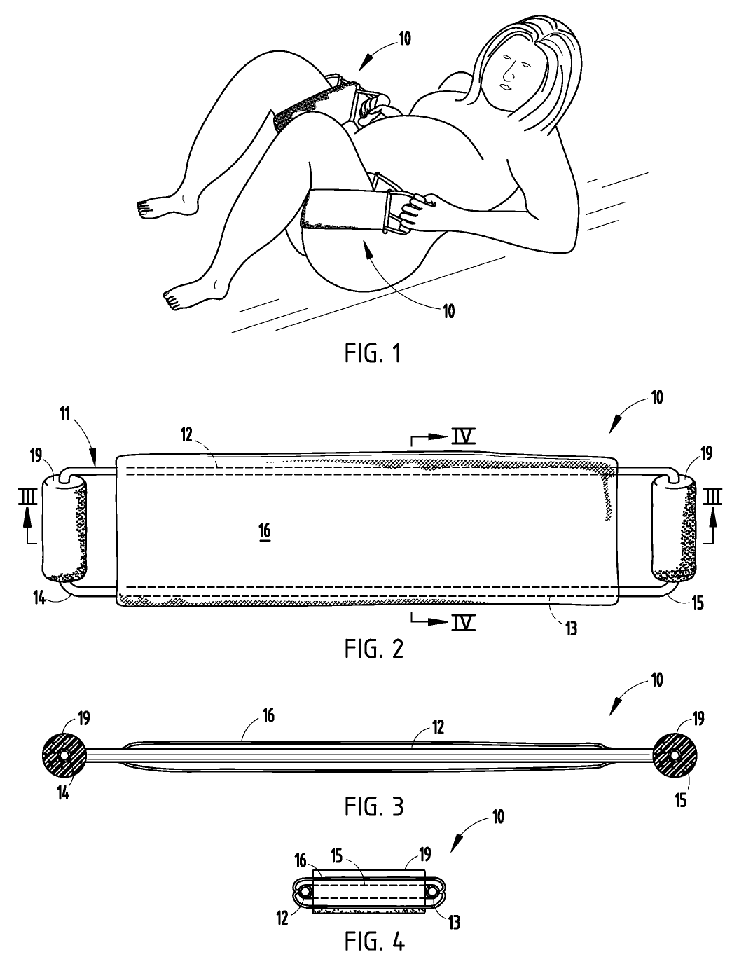

A birthing aid 10 (FIG. 1) includes a continuous band of resilient stretchable material 11 including opposing intermediate sections 12, 13 forming a leg sling, and opposing end sections 14, 15 forming integral loop handles on ends of the leg sling. A soft sheet cover 16 covers the intermediate sections 12, 13. By this arrangement, the birthing aid 10 is extremely well adapted to comfortably engage and resiliently support a mother's thigh and leg, while also letting the mother focus on child delivery. Further, when properly used, the child delivery is greatly assisted by the birthing aid 10 both by helping to properly position the mother's pelvic bones, and also causing the mother to naturally focus on appropriate muscle action (often called “pulling” versus “pushing”).

FIG. 1 shows a patient 1 grasping the handles 14, 15 of the birthing aid 10 to assist her in drawing up her legs as far as comfortably possible. This figure further depicts the location of the compliant band 12, 13, as it is wrapped about the patient's thigh area. While enduring childbirth, there is a natural tendency for a patient 1 who is experiencing an uncontrollable urge to push, to assume a supine or semi-supine position. The present aid 10 enables her to hold her head up, curl her spine, and draw her legs toward her body. In this manner, pressure from the contractions the patient 1 is experiencing is most efficiently transmitted to the birth canal. To assume this attitude, the patient 1 uses her hands to draw her legs as close to her body as possible, subject to the restraint imposed by her protuberant abdomen. During the exhausting childbirth process, the patient 1 often has difficulty in maintaining a grip on her legs. The present aid 10 helps considerably. Notably, the present device can also be used to “practice” childbirth, such as in classes for pregnant women.

In particular, by having straps that are not fixed and by providing straps that are resilient and have some “give,” the mother's arms are much less apt to get fatigued. While pulling, the biceps, deltoids and numerous forearm muscles are not fixed into one position. The amount of give in the straps allows for more complete and gradual flexion of these muscle groups. Also, in between contractions, the “give” in the straps allows for a more gradual rest for the arms and shoulders as compared to having a fixed material. Also, the use of the these straps allow for a more controlled flexion of the stemocleidomastoid in the neck, the pectoralis major and minor of the chest and facilitates the controlled use of the latissimus dorsi and trapezius of the upper back. The abdominal muscles are helped by the increased efficiency of the above mentioned groups of muscles, therefore making the delivery process more controlled and more efficient.

This birthing aid 10 is made to be particularly inviting to the mother/user, including appropriately soft material for the cover 16 (such as fleece, flannel, Velcro-covered material, soft cotton, or other environment-friendly soft materials . . . many of which are commonly associated with baby blankets). The cover 16 can also include aesthetic designs and/or have a soft color most suited for soothing the mother during childbirth. The illustrated cover 16 is sewn in place to form a cloth tubular sheet around the sling intermediate sections 12, 13. It is contemplated that the cover 16 can be permanently held in position, or can be made removable (by slipping off an end or by releasable snaps or Velcro® patches), such as to facilitate washing it.

The resilient stretchable material 11 is preferably a stretchy surgical tube or other stretchy bungee-cord-type material, such as is often used for physical exercise and/or physical therapy (or for stretch tie-downs). The illustrated stretch material is sufficiently strong to lift the mother's leg, such as by providing a lifting weight of 5 to 10 pounds force upon about 6 inches stretch. Notably, different strength stretch materials can be used for mother's needing different levels of lift force. The present material is a stretchable tube, however it is contemplated that other stretchy materials can be used, such as elastic fabrics. Further, it is contemplated that the present invention with stretch material can include a panel of material with loop ends, instead of a tubular stretchable cord or cable.

An advantage of the present arrangement using the resilient cord 11 is that it allows the mother in labor to hook her arms around the bands, thus giving the advantage of resting one's hands while insuring that progress made won't be lost. Another advantage is that it is easily placed under the mother's legs and grasped with a single, easy, swing-under-and-grip motion, either by the mother herself or by a helper. Typically, it does not require a multi-stage process nor cumbersome step to install it on the mother's leg in a given orientation, nor does it typically require providing assistance to help the mother grasp (and re-grasp) its handles. Also, even though the cord 11 and material 16 relax when the mother lessens her pulling force (such as between contractions), the material 16 tends to stay in place on the associated mother's leg, such that it remains ready for the next event.

In a preferred form, a foam tube 19 can be placed over end sections 12, 13 to increase surface area and reduce concentrated stress on the mother's hands. Similarly, the cover 16 also increases surface area and reduces concentrated stress on the mother's thigh and legs. Notably, one aid 10 is used on each leg.

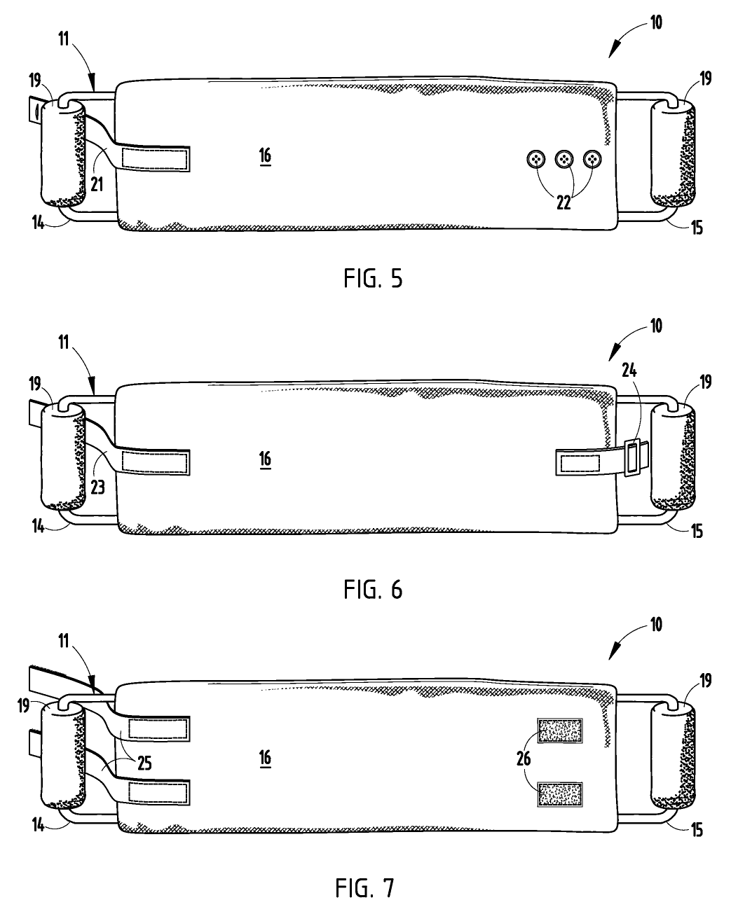

It is contemplated that the end sections 12, 13 can be integrated to form a single handle or can be connected in a way to form a single handle (e.g., by a connection means of apertured straps 21 and buttons 22 (FIG. 5), straps 23 and buckles 24 (FIG. 6), or straps 25 and Velcro® patches (FIG. 7). Alternatively, each of the mother's hands can simultaneously grasp the two end sections 12, 13 on each of two birthing aids 10 (i.e., one on each leg) with the aids 10 extending as slings around the mother's legs.

The present birthing aid is relatively low cost, simple to manufacture, and can be made of recyclable materials. It is contemplated that the birthing aid 10 can be thrown away after use or given to each birthing mother. Alternatively, it is contemplated that the cover 16 is removable and washable. Alternatively, the entire aid 10 is washable.

It is to be understood that variations and modifications can be made on the aforementioned structure without departing from the concepts of the present invention, and further it is to be understood that such concepts are intended to be covered by the following claims unless these claims by their language expressly state otherwise.

The embodiments of the invention in which an exclusive property or privilege is: claimed are defined as follows:

1. A birthing aid comprising:
a continuous band of resilient stretchable material including opposing intermediate sections forming a leg sling, and opposing end sections forming integral loop handles on ends of the leg sling; and
a soft sheet cover covering the intermediate section, the band and sheet cover being adapted to engage and support a mother's thigh and leg.

2. The birthing aid defined in claim 1, wherein the soft sheet cover includes a fabric material.

3. The birthing aid defined in claim 1, wherein the soft sheet cover is replaceable.

4. The birthing aid defined in claim 1, wherein the soft sheet cover has a color different than the band and is selected to be calming to a mother giving birth.

5. The birthing aid apparatus including a first one and a second one of the birthing aids defined in claim 1, the first and second ones being adapted for use on right and left legs of a birthing mother.

6. A birthing aid comprising:
a band of resilient stretchable material including at least one intermediate section forming a leg sling, and end sections forming handles on ends of the leg sling; and
a tubular soft sheet cover covering the at least one intermediate section and adapted to engage and support a mother's thigh and leg.

7. A birthing aid comprising:
a band of resilient stretchable material including at least one intermediate section forming a leg sling, and end sections forming handles on ends of the leg sling; and
a removable soft sheet cover covering the at least one intermediate section and adapted to engage and support a mother's thigh and leg.

8. The birthing aid defined in claim 7, wherein the removable soft sheet cover is either washable or disposable.

9. A birthing aid comprising:
a continuous band of resilient stretchable material including an intermediate section forming a leg sling, and end sections forming loops on ends of the leg sling; and
soft material wrapped around the loops to form soft handles providing maximum comfort to a mother in labor and that is using the birthing aid.

10. A method of practicing childbirth, comprising steps of:
providing a birthing aid having a resilient stretchable material with an intermediate section forming a leg sling and end sections forming handles;
placing the birthing aid under a birthing mother's thigh; and
pulling on the end sections in a manner causing the resilient stretchable material to stretch and provide assistance force to hold up the birthing mother's thigh and leg.

11. The method defined in claim 10, including placing a sheet cover on the intermediate section to add comfort and distributed support to the mother's thigh and leg.
